(19) United States
(12) Patent Application Publication
(10) Pub. No.: US 2021/0307619 A1
(43) Pub. Date: Oct. 7, 2021

(54) FEVER DETECTION

(71) Applicant: MP High Tech Solutions Pty Ltd, Eveleigh (AU)

(72) Inventors: Marek Steffanson, Mosman (AU); Gilad Francis, North Ryde (AU); Gabrielle de Wit, Pymble (AU); Daniel Petrov, Lilyfield (AU)

(21) Appl. No.: 17/201,900

(22) Filed: Mar. 15, 2021

Related U.S. Application Data
(60) Provisional application No. 63/005,085, filed on Apr. 3, 2020.
(60) Provisional application No. 63/006,005, filed on Apr. 6, 2020.

(51) Int. Cl. A61B 5/01; H04N 5/33; G06K 9/00; G01J 5/00; A61B 5/00

(52) U.S. Cl. CPC A61B 5/015; H04N 5/33; G06K 9/00255; G01J 5/0025; A61B 5/0077; A61B 2576/00; A61B 5/7264; A61B 5/746; A61B 5/7425; A61B 5/6844; G01J 2005/0077; A61B 5/0035

(57) ABSTRACT

Systems, methods, and apparatuses to detect persons having fever. For example, A fever scanner can have a thermal camera to capture a thermal image of a person, a distance sensor to measure a distance between the person and the fever scanner, and a processor to determine a first temperature from the thermal image and calculate a second temperature of the person based on the first temperature and the distance. When the second temperature is above a threshold, the fever scanner can generate an alert.

### RELATED APPLICATIONS

The present application claims the benefit of the filing dates of Prov. U.S. Pat. App. Ser. No. 63/005,085, filed Apr. 3, 2020, and Prov. U.S. Pat. App. Ser. No. 63/006,005, filed Apr. 6, 2020, both entitled “Fever Detection”, the entire disclosures of which applications are hereby incorporated herein by reference.

The present application relates to U.S. patent application Ser. No. 16/919,722, filed Jul. 2, 2020, published as U.S. Pat. App. Pub. No. 2021/0014396 on Jan. 14, 2021, and entitled “Hybrid Cameras,” the entire disclosure of which application is hereby incorporated herein by reference.

### FIELD OF THE TECHNOLOGY

At least some embodiments disclosed herein relate to temperature measurement in general and more particularly but not limited to the detection of persons having fever.

### BACKGROUND

Infrared radiation from a person corresponds to heat dissipation and temperature of the body of the person. Thus, thermal imaging of infrared radiation can be used to measure temperature.

There are different types of thermal imaging techniques. For example, U.S. Pat. No. 9,851,256, issued on Dec. 26, 2017 and entitled “Apparatus and method for electromagnetic radiation sensing”, discloses a thermal imaging device that uses micromechanical radiation sensing pixels to measure the intensity of infrared radiation in different locations of a thermal image. Such a thermal imaging device can have adjustable sensitivity and measurement range and can be utilized for human detection, fire detection, gas detection, temperature measurements, environmental monitoring, energy saving, behavior analysis, surveillance, information gathering and for human-machine interfaces, etc.

### BRIEF DESCRIPTION OF THE DRAWINGS

The embodiments are illustrated by way of example and not limitation in the figures of the accompanying drawings in which like references indicate similar elements.

FIG. 1 shows a fever scanner configured according to one embodiment.

FIGS. 2-4 illustrate a user interface for fever scanning according to one embodiment.

FIG. 5 shows a method implemented in a fever scanner according to one embodiment.

FIG. 6 shows an example of a dataset illustrating the relation between the temperature determined from a thermal image and the distance between a face and the thermal camera.

FIG. 7 shows an example of a dataset illustrated the relation between the size of a face recognized in an optical image and the distance between the face and the optical camera.

### DETAILED DESCRIPTION

The following description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

At least one embodiment disclosed herein includes a fever scanner that can be used to scan a person within a distance of 0.5 to 1.5 meters and determine whether the person has a fever. For example, the fever scanner can be positioned on a table in a reception area to scan a visitor. The scanner can be configured with an accuracy sufficient to determine whether the visitor has a fever corresponding to a typical symptom of an infectious disease, such as COVID-19, SARS, MERS, flu, etc. Fever can be detected without bringing the scanner in close proximity to the forehead of the visitor and thus avoid socially intrusive actions that can make the visitor uncomfortable.

The fever scanner can be implemented using a combination of a thermal camera and an optical camera. For example, a hybrid camera as disclosed in Prov. U.S. Pat. App. Ser. No. 62/871,660, filed Jul. 8, 2019 and entitled “Hybrid Cameras”, can be used, the entire disclosure of which is hereby incorporated herein by reference.

Such a fever scanner can be affordable and mass deployable, plug and play, with the accuracy of within half degree Celsius or Kelvin in body temperature measurements, without requiring a reference blackbody calibration source.

The high accuracy can be achieved for measuring the body temperature of a person (e.g., visitor) being scanned at varying distances by using an empirical formula to correct the measurement obtained by a thermal camera based on the distance between the person and the scanner. The distance can be measured based on an optical image of the face of the visitor. For example, a correction factor can be added to the temperature measurement calculated based on the thermal image of the facial portion of the visitor. The correction factor can be an empirical function of a distance between the scanner and the visitor. In one implementation, the correction factor in Celsius or Kelvin is proportional to (e.g., equal to) the distance in meters.

The distance between the visitor and the scanner can be measured using an optical camera that captures the facial image of the visitor in visible lights. The optical camera can have a resolution substantially higher than the resolution of the thermal camera. Thus, the image generated by the optical camera can be analyzed to determine the face size captured in the image. The face size captured in the image can be used to calculate a distance between the optical camera and the visitor and thus the distance between the scanner and the visitor.

Optionally, the distance can be measured using an alternative technique, such as an ultrasound sensor, a 3D depth camera, or another distance sensor.

For example, a microprocessor controller can be configured in the fever scanner to calculate the temperature of a visitor from the thermal image of the visitor, detect a face in an optical image of the visitor, calculate a distance between the visitor and the scanner/thermal camera, and correct the temperature calculated from the thermal image based on the distance. When the corrected temperature is above a threshold, the fever scanner can generate an alert.

In some implementations, the detected face in the optical image is used to select an area in the thermal image that corresponds to the face of the visitor to calculate the temperature of the visitor.

In some implementations, the thermal image sensor is configured with a resolution that is sufficient to estimate the distance between the visitor and the scanner. Thus, the optical camera can be omitted in such implementations.

Optionally, a display is presented to guide the visitor to a position for optimal temperature measurement. For example, the optical image of the visitor can be presented with an outline that identifies the expected boundary of the image of the head and shoulders of the visitor when the visitor is in an ideal position and/or distance from the scanner (e.g., 0.5 meter at the center of the view field of the scanner). When the optical image of the visitor partially fills in the outline, the outline superimposed on the optical image indicates that the visitor is off from the center of the view field and/or is too far from the scanner. Thus, the visitor can adjust his/her position for an improved measurement.

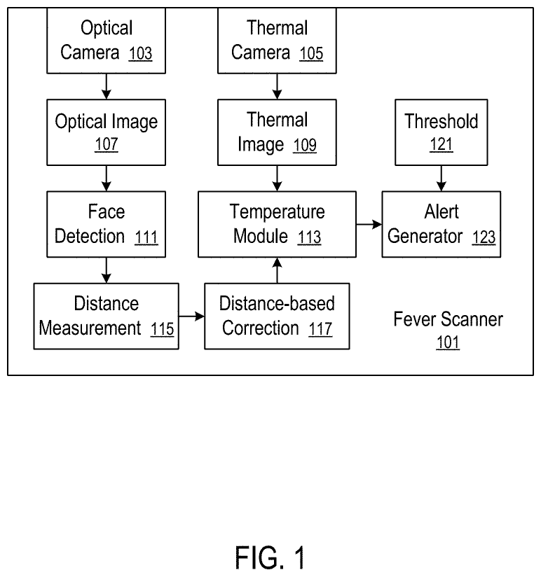

FIG. 1 shows a fever scanner configured according to one embodiment.

The fever scanner (101) of FIG. 1 has an optical camera (103) and a thermal camera (105). The field of view of the optical camera (103) and the field of view of the thermal camera (105) overlap substantially with each other such that the face of a person being scanned is captured in both the optical image (107) generated by the optical camera (103) and the thermal image (109) generated by the thermal camera (105). The optical camera (103) senses the lights visible to human eyes; and the thermal camera (105) senses infrared radiation from the body of the person.

The fever scanner (101) includes a face detection module (111) that identifies the face captured in the optical image (107). A distance measurement module (115) computes a distance between the scanner and the person. For example, an artificial neural network (ANN) can be trained to recognize the face/head portion in the optical image (107) and provides a distance between the scanner and the person having the face/head in the optical image (107).

For example, images of persons of different characteristics can be collected with distances measured using another method (e.g., measuring tapes) can be used to train the ANN to predict the measured distances.

Alternatively, after the face detection module (111) identifies a face portion in the optical image (107) and/or its boundary, a size of the face portion can be calculated (e.g., based on a bounding box the extracted face portion or an area measurement of the face portion in the optical image (107)). A formula can be used to convert the size to the distance between the face and the scanner.

The thermal image (109) includes a corresponding facial portion of the person being scanned. In some configurations, the person is instructed to be positioned with a background having a temperature that is substantially lower than the body temperature of a person. Thus, the facial portion can be extracted to calculate a temperature of the person from the radiation intensity of the facial portion.

Optionally, the location of the facial portion in the optical image (107) can be used to identify the corresponding facial portion in the thermal image (109) to calculate a temperature of the person.

The temperature module (113) of the fever scanner (101) is configured to not only calculate the temperature based on the infrared radiation intensity in the thermal image (109), but also adjust the calculated temperature to include a distance-based correction (117). For example, the distance-based correction (117) can be computed from the distance between the face being scanned and the fever scanner (101) based on an empirical formula.

The fever scanner (101) can include an alert generator (123) that compares the output of the temperature module (113) with a threshold (121). When the face temperature is above the threshold (121), the alert generator (123) can provide an indication that fever is detected.

At least some of the computing modules (e.g., 111, 115, 113) in the fever scanner (101) can be implemented via a microprocessor or controller executing instructions. Alternatively, or in combination, some of the computing modules (e.g., 111, 113, 115) can be implemented via logic circuits (e.g., using afield-programmable gate array (FPGA) or, an application-specific integrated circuit (ASIC)).

In some embodiments, the fever scanner (101) is enclosed within a housing and configured to be used as a standalone device. In other embodiments, the fever scanner (101) includes a communication port and/or a wireless connection that can be used to connect the optical image (107), the thermal image (109) to an external display device and/or an external data storage and processing location.

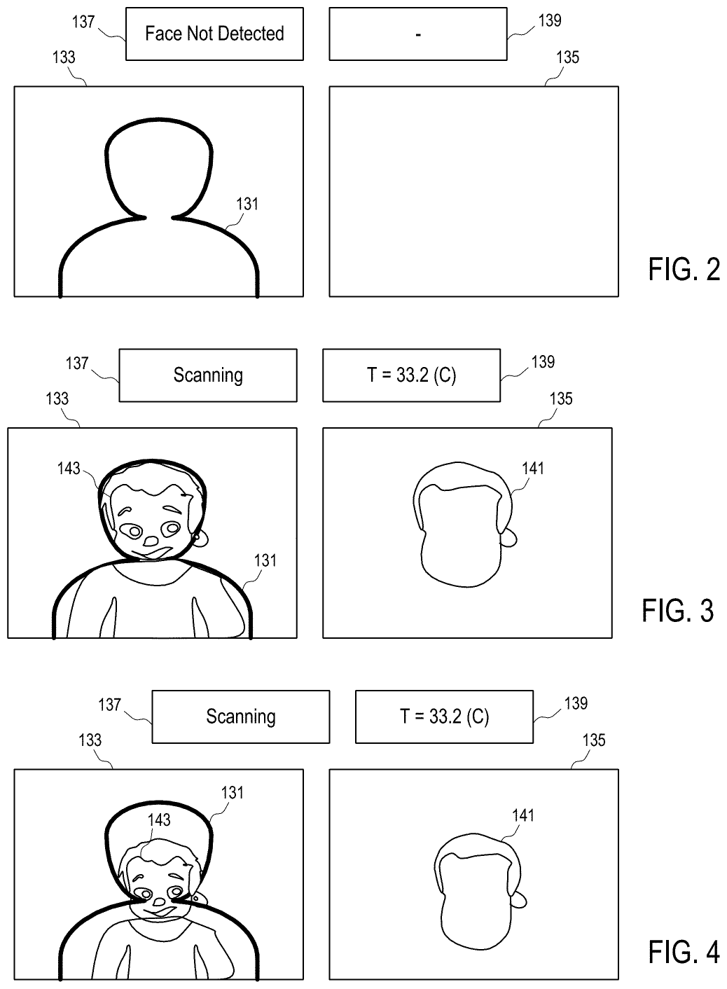

FIGS. 2-4 illustrate a user interface for fever scanning according to one embodiment.

The user interface includes a panel (133) configured to display the optical image (107) captured by the optical camera (103) of a fever scanner (101) and another panel (135) configured to display the thermal image (109) captured by the thermal camera (105) of the fever scanner (101).

Further, the user interface includes an area (137) configured to present the operation status of the fever scanner (101) and another area (139) configured to present the temperature of a person being scanned.

FIG. 2 illustrates a situation where no person is detected in the images (107 and 109). A predetermined outline (131) is presented in the panel (133) to indicate the preferred size and position of a person being scanned in the optical image (107).

FIG. 3 illustrates a situation where a person (143) is shown in the optical image (107) with a thermal image (141) of the person (143). Since the person (143) is an optimal distance (e.g., 0.5 meter) from the fever scanner (101), the outline of the person (143) in the optical image (107) substantially coincided with the predetermined outline (131).

FIG. 4 illustrates a situation where the person (143) is positioned from the fever scanner (101) at a distance (e.g., 1.2 meter) that is greater than the optimal distance (e.g., 0.5 meter). Thus, the outline of the person in the optical image (107) is substantially smaller than the predetermined outline (131). Based on the size difference, the fever scanner (101) computes a distance offset between the positions illustrated in FIG. 3 and FIG. 4 and corrects, based on the distance offset, the temperature determined from the radiation intensity of the thermal image (141) of the person (143). Thus, although the radiation intensity of the thermal image (141) in FIG. 4 is lower than that in FIG. 3, the fever scanner (101) can calculate the substantially same temperature for the person (143).

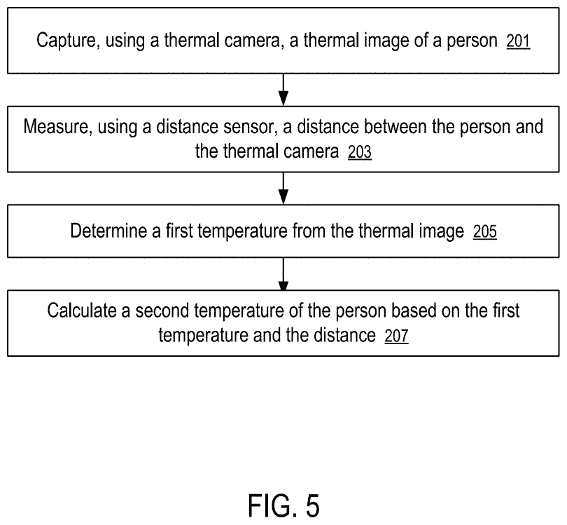

FIG. 5 shows a method implemented in a fever scanner according to one embodiment. For example, the method of FIG. 5 can be implemented in the fever scanner (101) of FIG. 1.

At block 201, a fever scanner (e.g., 101) captures, using a thermal camera (105), a thermal image (109) of a person.

At block 203, the fever scanner (e.g., 101) measures, using a distance sensor, a distance between the person and the thermal camera (105) of the fever scanner.

For example, the distance sensor can include a 3D depth camera to measure the distance, or an ultrasound generator to determine the distance based on a round trip time of an ultrasound signal.

For example, the distance sensor can include an optical camera (103) configured to generate an optical image (107) of the person based on sensing lights visible to human eyes and reflected from the person. The thermal camera is configured to generate the thermal image by sensing intensity of infrared radiation from the face, head and/or neck of the person.

At block 205, the fever scanner (e.g., 101) determines a first temperature from the thermal image (109).

At block 207, the fever scanner (e.g., 101) calculates a second temperature of the person based on the first temperature and the distance.

For example, the first temperature is based on the intensity of the infrared radiation; and the second temperature is calculated based on an empirical formula as a function of the distance. The empirical formula provides a difference between the first temperature and the second temperature; and the difference can be a linear function of the distance.

For example, after the optical camera (103) captures the optical image (107) of the person, a face detection module (111) recognizes a face portion of the person in the optical image and determine the distance based on the face portion.

For example, the face portion of the person in the optical image can be identified using an artificial neural network (ANN). A size of the face portion in the optical image (107) can be used to calculate the distance. Alternatively, the artificial neural network (ANN) can be trained to calculate the distance based on the size and characteristics of the face portion in the optical image (107). Thus, the optical image (107) can be used as an input to the artificial neural network to directly obtain the distance.

Optionally, the fever scanner (101) can have a user interface configured to provide an alert when the second temperature is above a threshold. The threshold can be adjusted to screening persons fora particular type of disease during an outbreak or pandemic. For example, the alert can be in the form of an audio signal (e.g., beep), or a visual indicator (e.g., flashing display of the second temperature).

Optionally, the user interface can be configured in a way as illustrated in FIGS. 2-4. The user interface configured to presents the optical image (107) with an outline (131) indicating a preferred location of an outline of the person in the optical image (107). Concurrently, the user interface further presents the thermal image (109), side by side with the optical image (107), in the user interface.

The fever scanner (101) can be enclosed within a housing adapted to position the fever scanner (101) at a fixed location facing a person (e.g., visitor) in vicinity of the location.

A processor can be configured within the housing of the fever scanner (101) to perform the methods discussed above by executing instructions. The instructions can be stored in a non-transitory machine readable medium such that when the instructions are executed by the processor the fever scanner (101) performs the methods discussed above.

Optionally, the processor can be configured in a data processing system located outside of the housing of the fever scanner (101). A wired or wireless connection between the data processing system to facilitate the computation discussed above. For example, the processor can be located in a personal computer or a server computer.

In one implementation, the resolution of the optical camera (103) is much greater than the resolution of the thermal camera (105). Thus, the optical image (107) can be used to identify a facial portion of the person and the corresponding portion in the thermal image (109) for an accurate determination of the first temperature. Alternatively, when the thermal camera (105) has a sufficient resolution of the recognition of the facial portion, the distance between the person and the fever scanner (101) can be measured based on the thermal image (109) instead of the optical image (107).

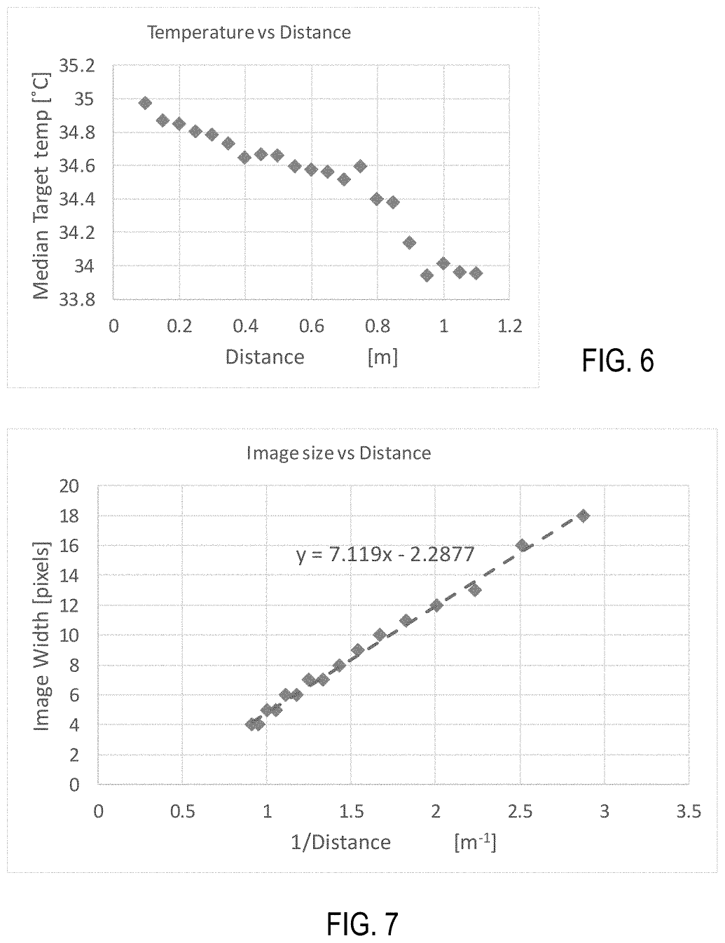

FIG. 6 shows an example of a dataset illustrating the relation between the temperature determined from a thermal image and the distance between a face and the thermal camera. The relation can be used to perform a distance-based correction of the temperature determined from a thermal image.

FIG. 7 shows an example of a dataset illustrated the relation between the size of a face recognized in an optical image and the distance between the face and the optical camera. The relation can be used to measure, using an optical camera (e.g., 103), the distance between a fever scanner (101) and a person being scanned for fever.

The present disclosure includes methods and apparatuses which perform the methods described above, including data processing systems which perform these methods, and computer readable media containing instructions which when executed on data processing systems cause the systems to perform these methods.

A typical data processing system can include an inter-connect (e.g., bus and system core logic), which interconnects a microprocessor(s) and memory. The microprocessor is typically coupled to cache memory.

The inter-connect interconnects the microprocessor(s) and the memory together and also interconnects them to input/output (I/O) device(s) via I/O controller(s). I/O devices can include a display device and/or peripheral devices, such as mice, keyboards, modems, network interfaces, printers, scanners, video cameras and other devices known in the art. In one embodiment, when the data processing system is a server system, some of the I/O devices, such as printers, scanners, mice, and/or keyboards, are optional.

The inter-connect can include one or more buses connected to one another through various bridges, controllers and/or adapters. In one embodiment the I/O controllers include a USB (Universal Serial Bus) adapter for controlling USB peripherals, and/or an IEEE-1394 bus adapter for controlling IEEE-1394 peripherals.

The memory can include one or more of: ROM (Read Only Memory), volatile RAM (Random Access Memory), and non-volatile memory, such as hard drive, flash memory, etc.

Volatile RAM is typically implemented as dynamic RAM (DRAM) which requires power continually in order to refresh or maintain the data in the memory. Non-volatile memory is typically a magnetic hard drive, a magnetic optical drive, an optical drive (e.g., a DVD RAM), or other type of memory system which maintains data even after power is removed from the system. The non-volatile memory can also be a random access memory.

The non-volatile memory can be a local device coupled directly to the rest of the components in the data processing system. A non-volatile memory that is remote from the system, such as a network storage device coupled to the data processing system through a network interface such as a modem or Ethernet interface, can also be used.

In the present disclosure, some functions and operations are described as being performed by or caused by software code to simplify description. However, such expressions are also used to specify that the functions result from execution of the code/instructions by a processor, such as a microprocessor.

Alternatively, or in combination, the functions and operations as described here can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

While one embodiment can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques can be carried out in a computer system or other data processing system in response to its processor, such as a microprocessor, executing sequences of instructions contained in a memory, such as ROM, volatile RAM, non-volatile memory, cache or a remote storage device.

Routines executed to implement the embodiments can be implemented as part of an operating system or a specific application, component, program, object, module or sequence of instructions referred to as “computer programs.” The computer programs typically include one or more instructions set at various times in various memory and storage devices in a computer, and that, when read and executed by one or more processors in a computer, cause the computer to perform operations necessary to execute elements involving the various aspects.

A machine readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. Further, the data and instructions can be obtained from centralized servers or peer to peer networks. Different portions of the data and instructions can be obtained from different centralized servers and/or peer to peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine readable medium in entirety at a particular instance of time.

Examples of computer-readable media include but are not limited to non-transitory, recordable and non-recordable type media such as volatile and non-volatile memory devices, Read Only Memory (ROM), Random Access Memory (RAM), flash memory devices, floppy and other removable disks, magnetic disk storage media, optical storage media (e.g., Compact Disk Read-Only Memory (CD ROM), Digital Versatile Disks (DVDs), etc.), among others. The computer-readable media can store the instructions.

The instructions can also be embodied in digital and analog communication links for electrical, optical, acoustical or other forms of propagated signals, such as carrier waves, infrared signals, digital signals, etc. However, propagated signals, such as carrier waves, infrared signals, digital signals, etc. are not tangible machine readable medium and are not configured to store instructions.

In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

In various embodiments, hardwired circuitry can be used in combination with software instructions to implement the techniques. Thus, the techniques are neither limited to any specific combination of hardware circuitry and software nor to any particular source for the instructions executed by the data processing system.

The above description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

In the foregoing specification, the disclosure has been described with reference to specific exemplary embodiments thereof. It will be evident that various modifications can be made thereto without departing from the broader spirit and scope as set forth in the following claims. The specification and drawings are, accordingly, to be regarded in an illustrative sense rather than a restrictive sense.

What is claimed is:

1. A fever scanner, comprising:
a thermal camera configured to capture a thermal image of a person; and
a distance sensor configured to measure a distance between the person and the fever scanner;
wherein the fever scanner is configured to determine a first temperature from the thermal image and calculate a second temperature of the person based on the first temperature and the distance.

2. The fever scanner of claim 1, wherein the distance sensor includes an optical camera configured to sense lights visible to human eyes and reflected from the person.

3. The fever scanner of claim 2, wherein the thermal camera is configured to sense intensity of infrared radiation from the person.

4. The fever scanner of claim 3, wherein the first temperature is based on the intensity of the infrared radiation; and the second temperature is calculated based on an empirical formula as a function of the distance.

5. The fever scanner of claim 4, wherein the empirical formula provides a difference between the first temperature and the second temperature; and the difference is a linear function of the distance.

6. The fever scanner of claim 2, wherein the optical camera is configured to capture an optical image of the person; and the distance sensor is configured to recognize a face portion of the person in the optical image and determine the distance based on the face portion.

7. The fever scanner of claim 6, wherein the face portion of the person in the optical image is identified using an artificial neural network.

8. The fever scanner of claim 6, wherein the distance sensor is configured to determine a size of the face portion in the optical image and calculates the distance based on the size of the face portion.

9. The fever scanner of claim 2, wherein the optical camera is configured to capture an optical image of the person; and the distance sensor is configured to determine the distance by applying the optical image as an input to an artificial neural network.

10. The fever scanner of claim 9, further comprising:
a user interface configured to provide an alert when the second temperature is above a threshold.

11. The fever scanner of claim 10, wherein the user interface is configured to present the optical image with an outline indicating a preferred location of an outline of the person in the optical image; and the user interface is further configured to present the thermal image concurrently with the optical image being presented in the user interface.

12. The fever scanner of claim 1, wherein the distance sensor includes a depth camera or an ultrasound generator.

13. A method, comprising:
capturing, using a thermal camera, a thermal image of a person; and
measuring, using a distance sensor, a distance between the person and the thermal camera;
determining a first temperature from the thermal image; and
calculating a second temperature of the person based on the first temperature and the distance.

14. The method of claim 13, wherein the measuring of the distance includes:
capturing, using an optical camera, an optical image of the person;
recognizing a face portion of the person in the optical image; and
determining the distance based on the recognizing of the face portion.

15. The method of claim 14, wherein the recognizing of the face portion is performed using an artificial neural network.

16. The method of claim 15, wherein the distance is an output from the artificial neural network.

17. An apparatus, comprising:
a housing adapted to position the apparatus at a fixed location facing a person arriving at a vicinity of the location;
a thermal camera configured on the housing, the thermal camera to generate a thermal image of the person;
an optical camera configured on the housing, the optical camera to generate an optical image of the person;
a processor configured to determine a distance between the apparatus and the person, determine a first temperature from the thermal image and a second temperature of the person based on the first temperature and the distance; and
a user interface configured to generate an alert when the second temperature is above a threshold.

18. The apparatus of claim 17, wherein the processor is configured within the housing; and the distance is using an artificial neural network.

19. The apparatus of claim 18, wherein a resolution of the optical camera is greater than a resolution of the thermal camera.

20. The apparatus of claim 19, wherein the first temperature is determined based on infrared radiation in an area identified according to a facial area of the person in the optical camera.
